(19) United States
(12) Patent Application Publication
(10) Pub. No.: US 2023/0172578 A1
(43) Pub. Date: Jun. 8, 2023

(54) ORAL X-RAY DEVICE HAVING ALIGNMENT FUNCTION

(71) Applicant: NANORAY CO., LTD., Daegu (KR)

(72) Inventors: Jae Yoon PARK, Gyeonggi-do (KR); Won SEONG, Daejeon (KR)

(21) Appl. No.: 17/919,602

(22) PCT Filed: May 11, 2021
(86) PCT No.: PCT/KR2021/005865

§ 371 Date: Oct. 18, 2022

(30) Foreign Application Priority Data
May 11, 2020 (KR) 10-2020-0056029

(51) Int. Cl. A61B 6/00; A61B 6/14; G01D 5/14

(52) U.S. Cl. CPC A61B 6/587; A61B 6/145; A61B 6/547; A61B 6/56; G01D 5/145

(57) ABSTRACT

An oral X-ray device having an alignment function includes an X-ray tube configured to generate and emit X-rays, and an X-ray receiver including a first gyro-sensor and a first communication module, having, at a corner thereof, a magnet configured to generate a magnetic force, and configured to receive the X-rays generated from the X-ray tube to acquire X-ray data about an object in the oral cavity. The X-ray tube includes a second gyro-sensor, an ultrasonic sensor configured to sense the X-ray receiver by generating ultrasonic waves and thus to sense a position of the X-ray receiver, and a proximity sensor configured to sense a distance between the X-ray tube and the X-ray receiver.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2021/005865, filed May 11, 2021, which claims priority to the benefit of Korean Patent Application No. 10-2020-0056029 filed in the Korean Intellectual Property Office on May 11, 2020, the entire contents of which are incorporated herein by reference.

### BACKGROUND

### 1. Technical Field

The present disclosure relates generally to an oral X-ray device having an alignment function. More specifically, the present disclosure relates to an oral X-ray device having an alignment function, in which an X-ray tube and an oral X-ray sensor device are each equipped with a gyro-sensor and the X-ray tube is equipped with a Hall sensor, an ultrasonic sensor, and a proximity sensor, so that radiography is performed in a state in which the oral X-ray sensor device is aligned.

### 2. Background Art

Generally, in order to perform intraoral radiography on a subject in a dental clinic, etc., an intraoral X-ray sensor is engaged with an instrument called a sensor holder and then is inserted into the subject's oral cavity. After that, X-rays are emitted onto the intraoral X-ray sensor from an X-ray irradiation device provided outside the subject's oral cavity, whereby an intraoral structure between the X-ray irradiation device and the intraoral X-ray sensor is radiographed.

Here, the intraoral X-ray sensor is an image sensor that generates electric signals that projection data of the subject is reflected on by sensing X-rays having passed through the subject, and the sensor holder is an assistant tool configured to place the intraoral X-ray sensor at a desired location inside the subject's oral cavity and to align the intraoral X-ray sensor and the X-ray irradiation device to face each other along a straight line when it is necessary. In this regard, Korean Patent Application Publication No. 2018-0086850 will be described in detail as follows.

Korean Patent Application Publication No. 2018-0086850 relates to a sensor holder of an intraoral X-ray sensor. According to this, the sensor holder of the intraoral X-ray sensor having a front surface for receiving X-rays and a rear surface opposite to the front surface and including a fastener protruding rearward from the rear surface includes: a grip fitted over a side surface of the fastener and surrounding at least a portion of the side surface; a coupler connected to the grip and including a support facing in the forward direction of the intraoral X-ray sensor while covering at least parts of the rear surface and a side surface of the intraoral X-ray sensor; a connector connected to the support and extending in the forward direction of the intraoral X-ray sensor; and a handle connected to the connector.

However, according to Korean Patent Application Publication No. 2018-0086850 described above, in order to perform horizontal, lateral, and distance alignments between the intraoral X-ray sensor and an X-ray tube, it is cumbersome for a practitioner to visually check the horizontal, lateral, and distance states between the tube and the sensor and then manually move the sensor or tube to align them.

In addition, according to the Korean Patent Application Publication No. 2018-0086850 described above, when the horizontal, lateral, and distance alignments are made visually and manually, there is a problem in that the image magnification rate in the vertical, horizontal, or diagonal direction of an image is changed, and a final image of teeth is distorted from the original one. This increases the possibility that the practitioner who performs treatment on the basis of such a distorted image may make a misdiagnosis.

### SUMMARY

An objective of the present disclosure is to provide an oral X-ray device having an alignment function, in which an X-ray tube and an oral X-ray sensor device are each equipped with a gyro-sensor and the X-ray tube is equipped with a Hall sensor, an ultrasonic sensor, and a proximity sensor, so that radiography is performed in a state in which the oral X-ray sensor device is aligned.

An oral X-ray device having an alignment function according to an aspect of the present disclosure includes: an X-ray tube configured to generate and emit X-rays; and

an X-ray receiver including a first gyro-sensor and a first communication module, having, at a corner thereof, a magnet configured to generate a magnetic force, and configured to receive the X-rays generated from the X-ray tube to acquire X-ray data about an object in an oral cavity.

The X-ray tube may include: a second gyro-sensor;

an ultrasonic sensor configured to sense the X-ray receiver by generating ultrasonic waves and thus to sense a position of the X-ray receiver; and

a proximity sensor configured to sense a distance between the X-ray tube and the X-ray receiver.

Furthermore, in the oral X-ray device having the alignment function, the X-ray tube may include a second communication module for bidirectional communication with the first communication module, and

the first communication module and the second communication module may use a Bluetooth or Wi-Fi type wireless short-range communication protocol.

Furthermore, in the oral X-ray device having the alignment function, the X-ray tube may include: an alignment check unit configured to check whether position, lateral, distance, and horizontal alignments between the X-ray tube and the X-ray receiver have been made; and

a display unit configured to display information of whether the position, lateral, distance, and horizontal alignments between the X-ray tube and the X-ray receiver have been made.

Furthermore, in the oral X-ray device having the alignment function, the alignment check unit may receive data sensed by the first gyro-sensor and data sensed by the second gyro-sensor, and determine whether the horizontal alignment between the X-ray receiver and the X-ray tube has been made.

Furthermore, in the oral X-ray device having the alignment function, the alignment check unit may sense the magnetic force generated from the magnet installed at the corner of the X-ray receiver to determine whether the lateral alignment of the X-ray receiver has been made.

Furthermore, in the oral X-ray device having the alignment function, the alignment check unit may sense the X-ray receiver through the ultrasonic waves emitted from the ultrasonic sensor to determine whether the position alignment of the X-ray receiver has been made.

Furthermore, in the oral X-ray device having the alignment function, the alignment check unit may sense the distance between the X-ray tube and the X-ray receiver through the proximity sensor to determine whether the distance alignment of the X-ray receiver has been made.

Furthermore, in the oral X-ray device having the alignment function, the X-ray tube may include a Hall sensor configured to sense the magnetic force generated from the magnet.

According to the embodiment of the present disclosure, a first gyro-sensor and a magnet are installed in an X-ray receiver, and a second gyro-sensor, a Hall sensor, an ultrasonic sensor, and a proximity sensor are installed in an X-ray tube, so that information of whether horizontal, lateral, position, and distance alignments of the X-ray receiver have been made is displayed on a display unit to allow a user to ascertain the information.

Furthermore, according to the embodiment of the present disclosure, an image having a constant magnification can be obtained by performing radiography in a state in which the X-ray receiver is aligned, and the amount of radiation exposure for the user can be minimized by preventing repeated re-radiography.

### BRIEF DESCRIPTION OF THE DRAWINGS

FIG. 1 is an exemplary view illustrating an oral X-ray device having an alignment function according to an embodiment of the present disclosure.

FIG. 2 is a block diagram illustrating the oral X-ray device having the alignment function according to the embodiment of the present disclosure.

### DETAILED DESCRIPTION

The present disclosure relates to an oral X-ray device having an alignment function, the device including: an X-ray tube generating and emitting X-rays; and

an X-ray receiver including a first gyro-sensor and a first communication module, having, at a corner thereof, a magnet generating a magnetic force, and receiving the X-rays generated from the X-ray tube to acquire X-ray data about an object in the oral cavity,

wherein the X-ray tube includes: a second gyro-sensor;

an ultrasonic sensor generating ultrasonic waves to sense the X-ray receiver and thus sensing the position thereof; and

a proximity sensor sensing the distance between the X-ray tube and the X-ray receiver.

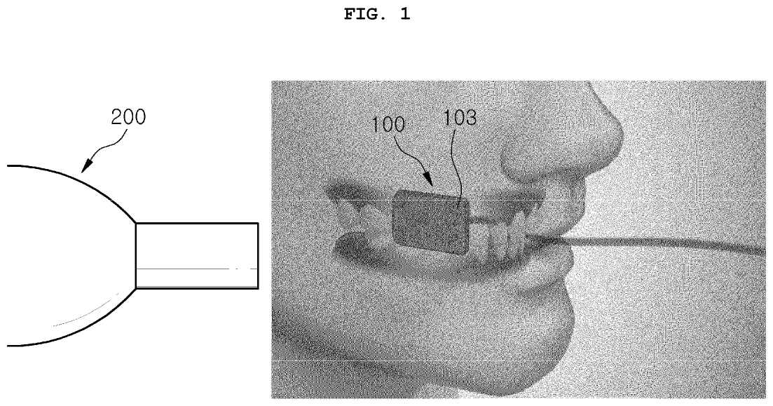

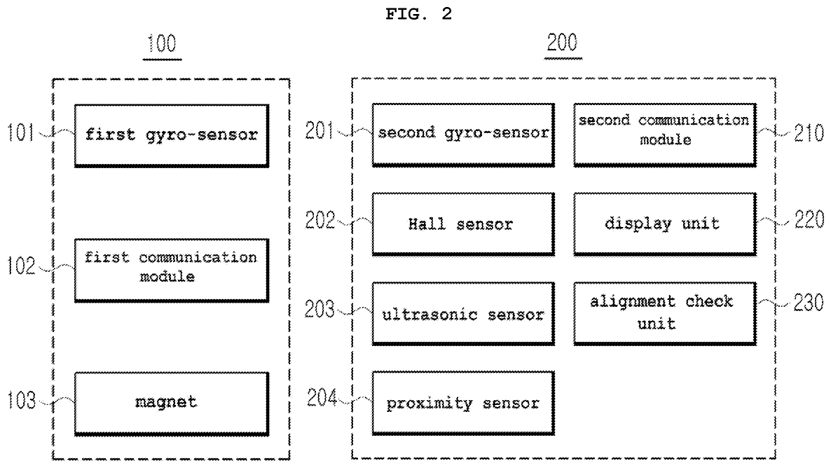

As illustrated in FIGS. 1 and 2, an oral X-ray device having an alignment function according to an embodiment of the present disclosure may include an X-ray receiver 100 and an X-ray tube 200.

The X-ray tube 200 is a device that generates X-rays and emits the X-rays onto the X-ray receiver 100, and may include a sensor unit, a second communication module 210, a display 220, and an alignment check unit 230.

The X-ray receiver 100 may acquire X-ray data of an object in the oral cavity by receiving X-rays generated from the X-ray tube 200, and transmit the acquired X-ray data to the X-ray tube 200 or a separate medical server (not illustrated) through the second communication module 210.

Here, a first communication module 102 and the second communication module 210 may be configured to enable bidirectional communication. The first communication module 102 and the second communication module 210 may establish bidirectional communication using a Bluetooth or Wi-Fi type wireless short-range communication protocol. The second communication module 210 may additionally establish a communication network using the medical server and the wireless short-range communication protocol.

Furthermore, the X-ray receiver 100 may have a polygonal shape, and may be configured to generate a magnetic force by having a magnet 103 at a corner thereof.

One or more magnets 103 may be installed at corners of the X-ray receiver 100, and the magnetic force thereof may be sensed by a Hall sensor 202 installed in the X-ray tube 200, so that the X-ray tube 200 may sense left and right sides of the X-ray receiver 100.

The X-ray tube 200 may further include: the sensor unit 201, 202, 203, and 204 collecting information of the X-ray receiver 100 for horizontal, distance, position, and lateral alignments of the X-ray receiver 100; the alignment check unit 230 checking whether the alignments have been made on the basis of the information collected by the sensor unit; and the display unit 220 displaying information determined by the alignment check unit 230.

The sensor unit may include a second gyro-sensor 201, the Hall sensor 202, an ultrasonic sensor 203, and a proximity sensor 204. A separate sensor may be additionally installed to collect information necessary for the horizontal, distance, position, and lateral alignments.

The first gyro-sensor 101 may collect information together with the second gyro-sensor 201 installed in the X-ray receiver 100 and transmit the collected data to the alignment check unit 230.

The alignment check unit 230 may determine whether the horizontal alignment between the X-ray receiver 100 and the X-ray tube 200 has been made on the basis of the information provided by the first gyro-sensor 101 and the second gyro-sensor 201, and display the determined information on the display unit 220.

The Hall sensor 202 may sense the magnetic force generated from the one or more magnets 103 installed at the corners of the X-ray receiver 100 to determine the left and right sides of the X-ray receiver 100. Specifically, each of the magnets 103 installed at each corner may be configured to form a different magnetic force or each of the magnets 103 may be configured to have a different shape to form a different magnetic force, so that the alignment check unit 230 may determine whether the left and right sides of the X-ray receiver 100 are aligned and display the determined information on the display unit 220.

The ultrasonic sensor 203 may sense the X-ray receiver 100 through ultrasonic waves emitted from the X-ray tube 200, and on the basis of the sensed information, the alignment check unit 230 may determine whether the position alignment of the X-ray receiver 100 has been made, and display the determined information on the display unit 220. Here, the X-ray tube 200 may further include an ultrasonic generator (not illustrated) that generates and emits ultrasonic waves.

The proximity sensor 204 may sense the distance between the X-ray tube 200 and the X-ray receiver 100, and on the basis of the sensed information, the alignment check unit 230 may determine whether the distance alignment has been made, that is, whether the distance between the X-ray tube 200 and the X-ray receiver 100 is located on an appropriate line, and display the determined information on the display unit 220.

In other words, the X-ray tube 200 may provide information necessary for the horizontal, distance, position, and lateral alignments of the X-ray receiver 100 to the alignment check unit 230 by using the second gyro-sensor 201, the Hall sensor 202, the ultrasonic sensor 203, and the proximity sensor 204, and the alignment check unit 230 may determine whether the alignment has been made and display the determined information on the display unit 220.

The display unit 220 may display the information determined by the alignment check unit 230, and additionally display information of the alignment which has been completed and information of the alignment which has not been completed, among the information of the horizontal, distance, position, and lateral alignments of the X-ray receiver 100.

Furthermore, the display unit 220 may display a message indicating that radiography is allowed to be performed when all the horizontal, distance, position, and lateral alignments are completed, and display a message indicating that the alignments have not been completed when the radiography is attempted in a state in which any one of the above four alignment criteria is not satisfied. These messages related to alignment completion or alignment mismatch may be controlled and managed by the alignment check unit 230.

Furthermore, the X-ray tube 200 according to the embodiment of the present disclosure is guided to perform the radiography only when all the four alignment criteria are satisfied, that is, the horizontal, distance, position, and lateral alignments, but is not limited thereto, and may be guided to align the X-ray receiver 100 by selecting at least any one of the four alignment criteria.

An oral X-ray device having an alignment function according to an embodiment of the present disclosure can be used in the medical field for intraoral radiography of patients.

1. An oral X-ray device having an alignment function, the oral X-ray device comprising:
an X-ray tube configured to generate and emit X-rays; and
an X-ray receiver comprising a first gyro-sensor, a first communication module, and a magnet positioned at a corner thereof configured to generate a magnetic force, the X-ray receiver configured to receive the X-rays generated from the X-ray tube to acquire X-ray data about an object in an oral cavity,
wherein the X-ray tube comprises:
a second gyro-sensor;
an ultrasonic sensor configured to sense the X-ray receiver by generating ultrasonic waves and thus to sense a position of the X-ray receiver; and
a proximity sensor configured to sense a distance between the X-ray tube and the X-ray receiver.

2. The oral X-ray device of claim 1, wherein the X-ray tube comprises a second communication module for bidirectional communication with the first communication module, and the first communication module and the second communication module use a Bluetooth or Wi-Fi type wireless short-range communication protocol.

3. The oral X-ray device of claim 1, wherein the X-ray tube comprises: an alignment check unit configured to check whether position, lateral, distance, and horizontal alignments between the X-ray tube and the X-ray receiver have been made; and a display unit configured to display information of whether the position, lateral, distance, and horizontal alignments between the X-ray tube and the X-ray receiver have been made.

4. The oral X-ray device of claim 3, wherein the alignment check unit receives data sensed by the first gyro-sensor and data sensed by the second gyro-sensor, and determines whether the horizontal alignment between the X-ray receiver and the X-ray tube has been made.

5. The oral X-ray device of claim 3, wherein the alignment check unit senses the magnetic force generated from the magnet installed at the corner of the X-ray receiver to determine whether the lateral alignment of the X-ray receiver has been made.

6. The oral X-ray device of claim 3, wherein the alignment check unit senses the X-ray receiver through the ultrasonic waves emitted from the ultrasonic sensor to determine whether the position alignment of the X-ray receiver has been made.

7. The oral X-ray device of claim 3, wherein the alignment check unit senses the distance between the X-ray tube and the X-ray receiver through the proximity sensor to determine whether the distance alignment of the X-ray receiver has been made.

8. The oral X-ray device of claim 1, wherein the X-ray tube comprises a Hall sensor configured to sense the magnetic force generated from the magnet.
